(19) United States
(12) Patent Application Publication
(10) Pub. No.: US 2021/0186749 A1
(43) Pub. Date: Jun. 24, 2021

(54) Thermal Therapy Garment Assembly

(71) Applicant: Christopher Kates, Mechanicsville, VA (US)

(72) Inventor: Christopher Kates, Mechanicsville, VA (US)

(21) Appl. No.: 16/722,030

(22) Filed: Dec. 20, 2019

(51) Int. Cl. A61F 7/02; A41D 27/20

(52) U.S. Cl. CPC A61F 7/02; A41D 27/202; A61F 2007/0238; A41D 2400/10; A61F 2007/0234; A41D 2400/32

(57) ABSTRACT

A thermal therapy garment assembly for includes a shirt that is wearable on a user. A plurality of pockets is each of the pockets is coupled to the shirt. A plurality of thermal packs is provided and each of the thermal packs is positionable in a respective one of the pockets. Each of the thermal packs is exposable to a heat source to apply heat therapy to strategic locations on the user's body. Each of the thermal packs is exposable to a cooling source to apply cold therapy to the strategic locations on the user's body. A plurality of straps is each coupled to the shirt. Each of the straps is tensionable along the shirt for compressing the shirt against the user's body. In this way each of the straps reduces blood circulation in the user's body for enhancing effectiveness of the heat or cold therapy.

### CROSS-REFERENCE TO RELATED APPLICATIONS

### Statement Regarding Federally Sponsored Research or Development

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to garment devices and more particularly pertains to a new garment device for applying heat therapy or cold therapy to strategic locations on a user's body.

### BRIEF SUMMARY OF THE INVENTION

An embodiment of the disclosure meets the needs presented above by generally comprising a shirt that is wearable on a user. A plurality of pockets is each of the pockets is coupled to the shirt. A plurality of thermal packs is provided and each of the thermal packs is positionable in a respective one of the pockets. Each of the thermal packs is exposable to a heat source to apply heat therapy to strategic locations on the user's body. Each of the thermal packs is exposable to a cooling source to apply cold therapy to the strategic locations on the user's body. A plurality of straps is each coupled to the shirt. Each of the straps is tensionable along the shirt for compressing the shirt against the user's body. In this way each of the straps reduces blood circulation in the user's body for enhancing effectiveness of the heat or cold therapy.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a back perspective view of a thermal therapy garment assembly according to an embodiment of the disclosure.

FIG. 2 is a front view of an embodiment of the disclosure.

FIG. 3 is a cross sectional view taken along line 3-3 of FIG. 2 of an embodiment of the disclosure.

FIG. 4 is a back view of an embodiment of the disclosure.

FIG. 5 is a perspective in-use view of an embodiment of the disclosure.

### DETAILED DESCRIPTION OF THE INVENTION

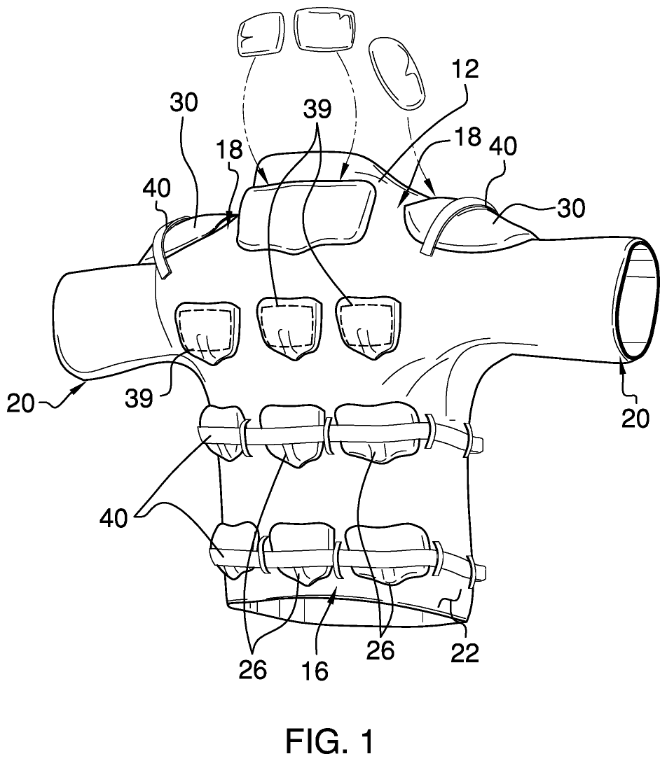

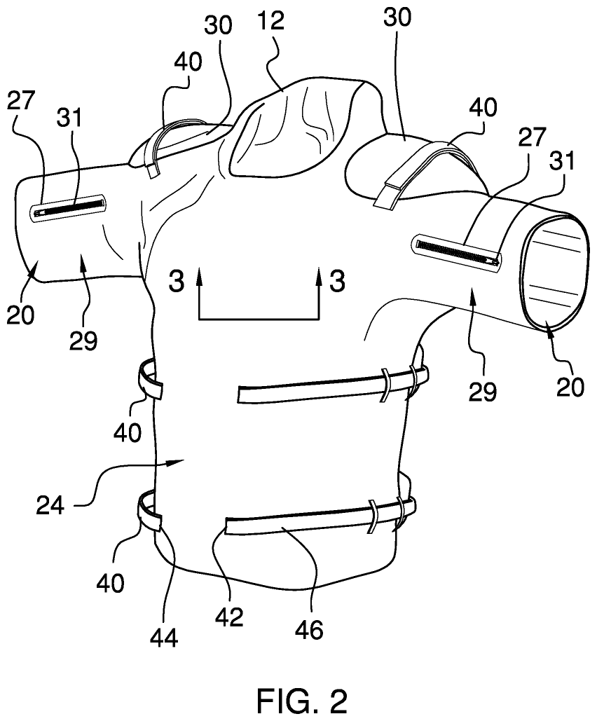

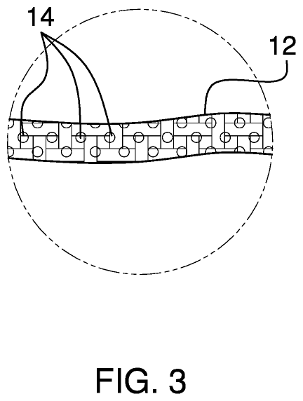

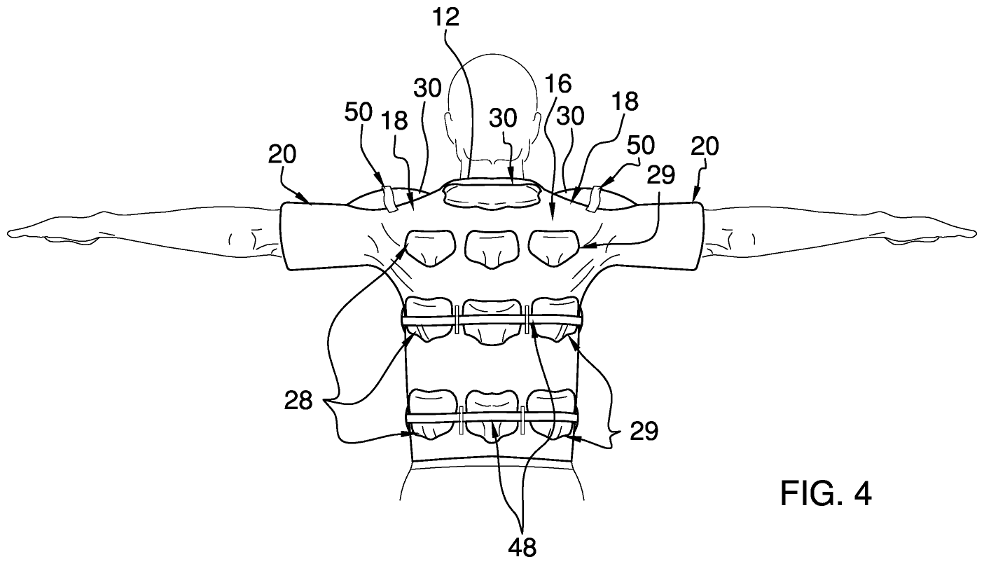

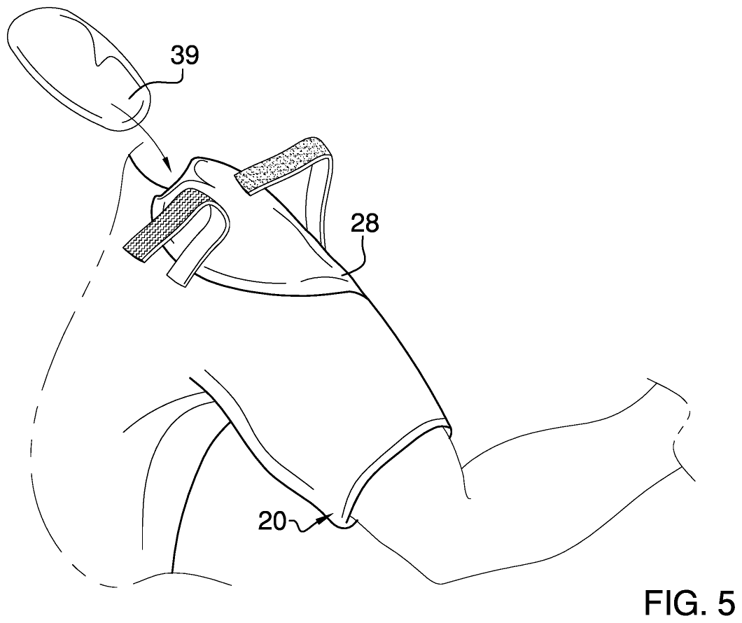

With reference now to the drawings, and in particular to FIGS. 1 through 5 thereof, a new garment device embodying the principles and concepts of an embodiment of the disclosure and generally designated by the reference numeral 10 will be described.

As best illustrated in FIGS. 1 through 5, the thermal therapy garment assembly 10 generally comprises a shirt 12 that is wearable on a user such that the shirt 12 is in thermal communication with the user's body. The shirt 12 may be comprised of a resiliently stretchable material such as spandex of the like. Additionally, the shirt 12 has copper filaments 14 integrated therein such that the copper filaments 14 facilitate pain relief in the user. The copper filaments 14 are distributed over an entirety of the shirt 12. The shirt 12 has a body portion 16, a pair of shoulder portions 18 and a pair of sleeves 20, and the shirt 12 has an outer surface 22 and a back side 24. The shirt 12 may be structured in the convention of a short sleeved tee shirt or the like.

A plurality of pockets 26 is each coupled to the shirt 12 and each of the pockets 26 is aligned with strategic points on the shirt 12 that correspond to locations on the user's body that would commonly have heat therapy or cold therapy applied thereto. Additionally, each of the pockets 26 is positioned on the outer surface 22 of the shirt 12. The plurality of pockets 26 includes a set of body pockets 28 that is each positioned on the back side 24 of the body portion 16 of the shirt 12. The set of body pockets 28 are arranged into a plurality of rows 29 on the body portion 16 of the shirt 12.

The plurality of pockets 26 includes a set of shoulder pockets 30 that is each positioned on an intersection between a respective one of the shoulder portions 18 of the shirt 12 and a respective one of the sleeves 20. The plurality of pockets 26 includes a set of sleeve pockets 32 that is each is positioned on a respective one of the sleeves 20. Each of the sleeve pockets 32 is positioned on a top side 34 of the respective sleeve 20. Additionally, each of the pockets 26 has a top end 36 and the top end 36 of each of the pockets 26 is open into an interior of the pockets 26. Each of the pockets 26 includes a flap 38 that is movably positioned on the shirt 12 and is aligned with the top end 36 of a respective one of the pockets 26. The flap 38 on each of the pockets 26 is foldable over the top end 36 of the respective pocket 26 for closing the top end 36 of the respective pocket 26.

Each of the sleeves 20 has a vent 27 extending therethrough for passing air into the sleeves 20. The vent 27 on each sleeve 20 is positioned on a front side 29 of the sleeve 20. A pair of closures 31, such as a zipper or other type of releasable closure, is coupled to a respective one of the sleeves 20. Each closure 31 is aligned with the vent 27 in the respective sleeve 20 for opening and closing the vent 27.

A plurality of thermal packs 39 is provided and each of the thermal packs 39 is positionable in a respective one of the pockets 26. Each of the thermal packs 39 is comprised of a thermally conductive material such that each of the thermal packs 39 is in thermal communication with the user's body when the thermal packs 39 are positioned in the respective pocket 26. Each of the thermal packs 39 is exposable to a heat source thereby facilitating each of the thermal packs 39 to absorb heat. In this way each of the thermal packs 39 can apply heat therapy to the strategic locations on the user's body.

Each of the thermal packs 39 is exposable to a cooling source thereby facilitating each of the thermal packs 39 to be cooled. In this way each of the thermal packs 39 can apply cold therapy to the strategic locations on the user's body. Each of the thermal packs 39 may be a gel thermal pack of any conventional design. Each of the thermal packs 39 is insertable through the top end 36 of the respective pocket 26. The flap 38 on the respective pocket 26 is foldable over the thermal pack 38 in the respective pocket 26 to retain the thermal pack 38 in the respective pocket 26.

A plurality of straps 40 is provided and each of the straps 40 is coupled to the shirt 12. Each of the straps 40 is tensionable along the shirt 12 for compressing the shirt 12 against the user's body. In this way each of the straps 40 can reduce blood circulation in the user's body for enhancing effectiveness of the heat or cold therapy. Each of the straps 40 is releasably matable to the shirt 12 when the straps 40 are tensioned along the shirt 12.

Each of the straps 40 has a first end 42, a second end 44 and a first surface 46 extending therebetween. The first surface 46 of each of the straps 40 is coupled to the outer surface 22 of the shirt 12 and each of the first 42 and second 44 ends of each of the straps 40 is free from the shirt 12. Each of the first 42 and second 44 ends of each of the straps 40 is matable to the outer surface 22 of the shirt 12. Additionally, each of the straps 40 may be comprised of an elastomeric material. In this way each of the straps 40 can be stretched fully around the shirt 12.

The plurality of straps 40 includes a set of body straps 48 and each of the body straps 48 is horizontally oriented on the shirt 12. Each of the body straps 48 is oriented collinear with a respective one of the rows 29 of the body pockets 28. The plurality of straps 40 includes a pair of shoulder straps 50 and each of the shoulder straps 50 is positioned on a respective one of the shoulder portions 18 of the shirt 12. Each of the shoulder straps 50 is aligned with an intersection of a respective one of the sleeves 20 and the respective shoulder portion. Moreover, each of the shoulder straps 50 extends across a respective one of the shoulder pouches 30. Each of the straps 40 may include hook and loop fasteners or the like for releasably engaging the shirt 12.

In use, the shirt 12 is worn when the user needs to apply heat therapy or cold therapy to selected areas of the user's upper body. Selected ones of the thermal packs 39 are heated or cooled, depending on the user's wishes for heat therapy or cold therapy. Each of the thermal packs 39 is positioned in a respective one of the pockets 26 when the thermal packs 39 have been heated or cooled. In this way the user can apply heat therapy or cold therapy to selected areas on the user's upper body. Additionally, the straps 40 can be tensioned along the shirt 12 for compressing the shirt 12 against the user's body. In this way blood circulation can be slowed for enhancing the effectiveness of the heat or cold therapy. The shirt 12 can be worn underneath clothing that the user is wearing or the shirt 12 can be worn by itself.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A thermal therapy garment assembly being configured to position hot or cold packs at strategic locations on a user's body for pain therapy, said assembly comprising:
a shirt being wearable on a user wherein said shirt is configured to be in thermal communication with the user's body, said shirt having copper filaments being integrated therein wherein said copper filaments are configured to facilitate pain relief in the user;
a plurality of pockets, each of said pockets being coupled to said shirt, each of said pockets being aligned with strategic points on the shirt corresponding to locations on the user's body that would commonly have heat therapy or cold therapy applied thereto;
a plurality of thermal packs, each of said thermal packs being positionable in a respective one of said pockets, each of said thermal packs being comprised of a thermally conductive material wherein each of said thermal packs is configured to be in thermal communication with the user's body when said thermal packs are positioned in said respective pocket, each of said thermal packs being exposable to a heat source wherein each of said thermal packs is configured to absorb heat thereby facilitating each of said thermal packs to apply heat therapy to the strategic locations on the user's body, each of said thermal packs being exposable to a cooling source wherein each of said thermal packs is configured to be cooled thereby facilitating each of said thermal packs to apply cold therapy to the strategic locations on the user's body; and
a plurality of straps, each of said straps being coupled to said shirt, each of said straps being tensionable along said shirt for compressing said shirt against the user's body wherein each of said straps is configured to reduce blood circulation in the user's body for enhancing effectiveness of the heat or cold therapy, each of said straps being releasably matable to said shirt when said straps are tensioned along said shirt.

2. The assembly according to claim 1, wherein:
said shirt has a body portion, a pair of shoulder portions and a pair of sleeves, said shirt having an outer surface and a front side; and
each of said pockets is positioned on said outer surface of said shirt;
said plurality of pockets includes a set of body pockets each being positioned on said front side of said body portion of said shirt, said set of body pockets being arranged into a plurality of rows on said body portion of said shirt;
said plurality of pockets includes a set of shoulder pockets each being positioned on a respective one of said shoulder portions;
said plurality of pockets includes a set of sleeve pockets each being positioned on a respective one of said sleeves, each of said sleeve pockets being positioned on a top side of said respective sleeve.

3. The assembly according to claim 2, wherein:
each of said pockets has a top end, said top end of each of said pockets being open into an interior of said pockets; and
each of said pockets includes a flap being movably positioned on said shirt and being aligned with said top end of a respective one of said pockets, said flap on each of said pockets being foldable over said top end of said respective pocket for closing said top end of said respective pocket.

4. The assembly according to claim 3, wherein each of said thermal packs is insertable through said top end of said respective pocket, said flap on said respective pocket being foldable over said thermal pack in said respective pocket to retain said thermal pack in said respective pocket.

5. The assembly according to claim 2, wherein each of said straps has a first end, a second end and a first surface extending therebetween, said first surface of each of said straps being coupled to said outer surface of said shirt, each of said first and second ends of each of said straps being free from said shirt, each of said first and second ends of each of said straps being matable to said outer surface of said shirt.

6. The assembly according to claim 5, wherein said plurality of straps includes a set of body straps, each of said body straps being horizontally oriented on said shirt, each of said body straps being oriented collinear with a respective one of said rows of said body pockets.

7. The assembly according to claim 6, wherein said plurality of straps includes a pair of shoulder straps, each of said shoulder straps being positioned on a respective one of said shoulder portions of said shirt, each of said shoulder straps being aligned with an intersection of a respective one of said sleeves and said respective shoulder portion.

8. A thermal therapy garment assembly being configured to position hot or cold packs at strategic locations on a user's body for pain therapy, said assembly comprising:
a shirt being wearable on a user wherein said shirt is configured to be in thermal communication with the user's body, said shirt having copper filaments being integrated therein wherein said copper filaments are configured to facilitate pain relief in the user, said shirt having a body portion, a pair of shoulder portions and a pair of sleeves, said shirt having an outer surface and a front side;
a plurality of pockets, each of said pockets being coupled to said shirt, each of said pockets being aligned with strategic points on the shirt corresponding to locations on the user's body that would commonly have heat therapy or cold therapy applied thereto, each of said pockets being positioned on said outer surface of said shirt, said plurality of pockets including a set of body pockets each being positioned on said front side of said body portion of said shirt, said set of body pockets being arranged into a plurality of rows on said body portion of said shirt, said plurality of pockets including a set of shoulder pockets each being positioned on a respective one of said shoulder portions, said plurality of pockets including a set of sleeve pockets each being positioned on a respective one of said sleeves, each of said sleeve pockets being positioned on a top side of said respective sleeve, each of said pockets having a top end, said top end of each of said pockets being open into an interior of said pockets, each of said pockets including a flap being movably positioned on said shirt and being aligned with said top end of a respective one of said pockets, said flap on each of said pockets being foldable over said top end of said respective pocket for closing said top end of said respective pocket;
a plurality of thermal packs, each of said thermal packs being positionable in a respective one of said pockets, each of said thermal packs being comprised of a thermally conductive material wherein each of said thermal packs is configured to be in thermal communication with the user's body when said thermal packs are positioned in said respective pocket, each of said thermal packs being exposable to a heat source wherein each of said thermal packs is configured to absorb heat thereby facilitating each of said thermal packs to apply heat therapy to the strategic locations on the user's body, each of said thermal packs being exposable to a cooling source wherein each of said thermal packs is configured to be cooled thereby facilitating each of said thermal packs to apply cold therapy to the strategic locations on the user's body, each of said thermal packs being insertable through said top end of said respective pocket, said flap on said respective pocket being foldable over said thermal pack in said respective pocket to retain said thermal pack in said respective pocket; and
a plurality of straps, each of said straps being coupled to said shirt, each of said straps being tensionable along said shirt for compressing said shirt against the user's body wherein each of said straps is configured to reduce blood circulation in the user's body for enhancing effectiveness of the heat or cold therapy, each of said straps being releasably matable to said shirt when said straps are tensioned along said shirt, each of said straps having a first end, a second end and a first surface extending therebetween, said first surface of each of said straps being coupled to said outer surface of said shirt, each of said first and second ends of each of said straps being free from said shirt, each of said first and second ends of each of said straps being matable to said outer surface of said shirt, said plurality of straps including a set of body straps, each of said body straps being horizontally oriented on said shirt, each of said body straps being oriented collinear with a respective one of said rows of said body pockets, said plurality of straps including a pair of shoulder straps, each of said shoulder straps being positioned on a respective one of said shoulder portions of said shirt, each of said shoulder straps being aligned with an intersection of a respective one of said sleeves and said respective shoulder portion.
